(19) United States
(12) Patent Application Publication
(10) Pub. No.: US 2010/0255040 A1
(43) Pub. Date: Oct. 7, 2010

(54) COMPOSITION FOR IMPROVING TLR-MEDIATED CELLULAR IMMUNITY COMPRISING POLY-GAMMA-GLUTAMIC ACID

(75) Inventors: Moon-Hee Sung, Daejeon (KR); Chul Joong Kim, Daejoen (KR); Haryoung Poo, Daejoen (KR); Il Han Lee, Gyeonggi-do (KR); Yang-Hyun Kim, Daejeon (KR)

Correspondence Address: MOORE & VAN ALLEN PLLC, P.O. BOX 13706, Research Triangle Park, NC, 27709, US

(73) Assignee: BIOLEADERS CORPORATION, Daejeon (KR)

(21) Appl. No.: 12/682,662

(22) PCT Filed: Oct. 15, 2007
(86) PCT No.: PCT/KR07/05027

§ 371 Date: Jun. 3, 2010

(30) Foreign Application Priority Data
Oct. 12, 2007 (KR) 10-2007-0103161

(51) Int. Cl. A61K 39/00; A61P 31/12; A61P 31/04; A61P 35/00

(52) U.S. Cl. 424/278.1

(57) ABSTRACT

The present invention relates to a composition for improving cellular immunity, which comprises an effective dose of poly-gamma-glutamic acid, and more particularly, to a composition for improving cellular immunity comprising poly-gamma-glutamic acid, which has effects of inducing enhancement of TLR-mediated Th1 cellular immunity and improving antigen presenting activity to maintain the improved activity. The inventive composition comprising an effective dose of poly-gamma-glutamic acid has very little toxicity and side effects, and can be added to a vaccine composition for preventing and treating animal viral or bacterial infections and cancer, or a vaccine composition for preventing and treating human viral or bacterial infections and cancer to show cellular immunity-enhancing effects.

### TECHNICAL FIELD

The present invention relates to a composition for improving cellular immunity, which comprises an effective dose of poly-gamma-glutamic acid, and more particularly, to a composition for improving cellular immunity comprising poly-gamma-glutamic acid, which has effects of inducing enhancement of TLR-mediated Th1 cellular immunity and improving antigen presenting activity to maintain the improved activity.

### BACKGROUND ART

The concepts of cellular immunity and humoral immunity are comparative, cellular immunity is an immune response that is induced by antigen-specific T cells or antigen-nonspecific NK cells (Natural Killer cells), and macrophages.

Humoral immune response is induced by antibodies which are not related to cells and thus suitable for removing antigens such as bacteria acting independently without association with cells. However, among antigens, there are antigens which are not present outside cells, but are associated with host cells, or are present inside the cells. For example, viruses are a representative example of proliferating in host cells, and cancer cells are generated by oncogenic transformation of cells themselves so that most antigens are associated with cells or present inside the cells. In these cases, problems inside the cells, where the antigens are present, should be solved to effectively remove the antigens, but antibodies cannot directly penetrate into cells so that it is hard for the antibodies to react with the antigens in the cells.

Although an antibody against a cell surface antigen shows cytotoxic reactions such as ADCC (antibody dependent cellular cytotoxicity) in which antibodies bind to antigens on cell surface and thus cells are phagocytosed by NK cells or macrophages, or complement mediated lysis, it is necessary to induce a cell mediated immune response in order to effectively remove antigens located inside the cells. Namely, it is possible to effectively remove cell-associated antigens by a cellular immune response in which cells containing specific antigens are eliminated.

Cellular immune responses are classified into two categories according to effector functions: antigen-specific immune response of effector T cell function and immune response of antigen-nonspecific effector cells such as natural killer cells or macrophages. Cytotoxic T cell (CTL)-mediated response is an immune response in which antigen specific CTL directly acts on cells containing antigen to kill the antigen-containing cells, which is one of typical specific immune responses. Although CD4 T cells, which mediate delayed type hypersensitivity response, among T cells, does not directly kill cells, as an effector cell (CD4 effector T cell) of a cell mediated immune response, they activate macrophages to promote the cell mediated immune response. In addition, NK cells or macrophages also play an important role in cell mediated immune responses, and although these cells are antigen-nonspecific cells, they can recognize allogeneic cells, tumor cells, virus-infected cells and the like to destroy them.

However, the stability of the most conventional immunoadjuvants used has not been secured, or the conventional immunoadjuvants are effective for enhancement of humoral immunity but they are not effective in preventing and treating viral infection diseases, cancer diseases, etc.

It has been reported that GlaxoSmithkline's preventive cervical cancer vaccine AS04 adjuvant shows partial effects of enhancing cellular immunity, but it is limitedly used for injectable vaccines, so that it cannot be used via oral route or mucosal route.

Most pathogens penetrate through the mucosal surface, and most infections primarily occur in mucosa and submucosal tissue. Thus, conventional parenteral vaccines are very ineffective in inducing a mucosal immune response and thus there have been considerable efforts to develop optimal mucosal immune system. As one of these efforts, immunoadjuvants (liposomes, immuno-stimulating complexes, and centrosomes) improving antigen delivery to immune cells in submucosal tissue have been developed and also it was reported that experiments were performed using the same (Sjolander et al, J. Leukocyte Biol. 64:713, 1998). However, although mucosal immunity is effective for many cases, it is necessary to combine mucosal immune system with non-mucosal (systemic) immune system in order to induce an effective immune response in many infections.

When considering the above mentioned techniques, mass production of selected antigenic material and the development of cost-effective adjuvants which can maximize the effect of antigenic material and safely deliver it, are required prerequisites, in order to develop a pharmaceutical composition which is commercially competitive and effective. In addition, immunoadjuvants, which can be transdermally, orally, mucosally and systemically administered and can modulate and focus immune responses, is necessary.

Accordingly, the present inventors have made extensive efforts to develop a more effective and safe immunoadjuvant, and as a result, found that, poly-gamma-glutamic acid produced by Bacillus strain has effects of enhancing TLR4-mediated Th1 cellular immunity, increases antigen presenting activity, and maintains the increased activity, and confirmed that poly-gamma-glutamic acid is useful as a CMI (cell-mediated immunity) adjuvant, and is safe upon oral administration, thereby completing the present invention.

### SUMMARY OF THE INVENTION

Therefore, it is a main object of the present invention to provide a composition for enhancing cellular immunity, which comprises an effective dose of poly-gamma-glutamic acid.

It is another object of the present invention to provide a vaccine composition for preventing and treating human viral and bacterial infections and cancer, or a vaccine composition for preventing and treating animal viral and bacterial infections, which comprises an effective dose of poly-gamma-glutamic acid, and has an effect of enhancing cellular immunity.

To achieve the above object, the present invention provides a composition for enhancing cellular immunity, which comprises an effective dose of poly-gamma-glutamic acid.

In addition, the present invention provides a vaccine composition for preventing and treating human viral or bacterial infections and cancer through enhancement of cellular immunity, which comprises an effective dose of poly-gamma-glutamic acid.

The present invention also provides a vaccine composition for preventing and treating animal viral or bacterial infections through enhancement of cellular immunity, which comprises an effective dose of poly-gamma-glutamic acid.

The above and other objects, features and embodiments of the present invention will be more clearly understood from the following detailed description and accompanying claims.

### BRIEF DESCRIPTION OF THE DRAWINGS

FIG. 1 is the results showing the effect of poly-gamma-glutamic acid on activation of bone marrow-derived macrophages by a toll-like receptor 4 (TLR4) signaling.

FIG. 2 is the results showing the effect of poly-gamma-glutamic acid on dendritic cell activation.

FIG. 3 is the measurement results showing the amounts of IL-12p70 which is secreted by dendritic cells differentiated from bone marrow cells of wild-type C3H/HeN mouse and TLR4 mutant C3H/HeJ mouse, treated with γ-PGA.

FIG. 4 shows distribution of CD8+ T cells secreting IFN-γ, obtained by FACS analysis in order to examine T cell activation according to the ratio between dendritic cells and T cells.

FIG. 5 is the results showing antigen presenting activity by analyzing T cell activation according to the ratio between dendritic cells and T cells.

FIG. 6 is the results showing the effect of γ-PGA treatment on suppression of TLR4-dependent signaling through inhibition of TLR4 expression by siRNA.

FIG. 7 is the results showing anticancer effects of a vaccine composition comprising E7 expressed on the Lactobacillus surface, using poly-gamma-glutamic acid as an enhancer of cellular immunity.

FIG. 8 is the results showing that a humoral immune response against human CEA is induced in mice by Lactobacillus casei expressing CEA on the surface thereof and a γ-PGA-containing composition.

FIG. 9 is ELISPOT assay results showing that a cellular immune response against human CEA is induced by Lactobacillus casei expressing CEA on the surface thereof and a γ-PGA-containing composition.

FIG. 10 is FACS assay results showing that a cellular immune response against human CEA is induced by Lactobacillus casei expressing CEA on the surface thereof and a γ-PGA-containing composition.

FIG. 11 is the results showing anticancer effects (tumor size decrease) by administration of Lactobacillus casei expressing CEA on the surface thereof and a γ-PGA-containing composition.

FIG. 12 is the results showing anticancer effects (survival rate increase) by administration of Lactobacillus casei expressing CEA on the surface thereof and a γ-PGA-containing composition.

### DETAILED DESCRIPTION OF THE INVENTION, AND PREFERRED EMBODIMENTS

In one aspect, the present invention relates to a composition for improving cellular immunity, which comprises an effective dose of poly-gamma-glutamic acid.

In the present invention, the molecular weight of poly-gamma-glutamic acid used for the composition for improving cellular immunity is preferably 100 kDa˜15,000 kDa, and more preferably 1,000 kDa˜15,000 kDa.

In the present invention, it was confirmed that the poly-gamma-glutamic acid induces enhancement of TLR-mediated Th1 cellular immunity by bone marrow-derived macrophages and dendritic cells, and improves antigen presenting-activities of dendritic cells and thus maintains the improved activities.

In the present invention, the composition for improving immunity is preferably added to a vaccine for preventing and treating human viral and bacterial infections or animal viral and bacterial infections to show immunity enhancement effects.

The composition for enhancing immunity comprising poly-gamma-glutamic acid as an effective ingredient, according to the present invention, can be formulated into oral dosing forms such as powders, granules, tablets, capsules, suspensions, emulsions, syrups, aerosols etc, external preparation, suppository and sterilized solution for injection according to a conventional preparation method therefore.

The carrier, excipient and diluent, that can be contained in the composition for enhancing immunity include lactose, dextrose, sucrose, sorbitol, mannitol, xylitol, erythritol, maltitol, starch, gum acaia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate and mineral oil.

The composition of the present invention can be formulated into a preparation form, together with a conventional diluent or excipient such as fillers, extenders, binders, wetting agents, disintegrants, surfactants etc. A solid preparation for oral administration includes tablets, pills, powders, granules, capsules etc, and the solid preparation is formulated by mixing the compound with at least one excipient, for example, starch, calcium carbonate, sucrose, lactose, gelatin and the like. Also, a lubricant such as magnesium stearate and talc is used in addition to the excipients. A liquid preparation for oral administration includes suspension, zipeprol, emulsion, syrup and the like, and various excipients, for example, wetting agents, flavoring agents, fragrances, preservatives etc, can be contained thereto in addition to conventional diluents such as water and liquid paraffin. A preparation for parenteral administration includes sterile aqueous solution, non-aqueous solution, suspensions, emulsions, a lyophilized preparation, and suppositories. Examples of non-aqueous solution and suspensions include vegetable oil such as propylene glycol and polyethylene glycol and olive oil, and injectable esters such as ethyloleate and the like. Basic materials of suppositories include witepsol, macrogol, tween 61, cacao butter, laurin butter, glycerol, gelatine and the like.

In the inventive pharmaceutical composition for enhancing immunity, which contains poly-gamma-glutamic acid as an effective ingredient, γ-PGA can be preferably added at different dose levels depending on patient's condition and weight, the disease state being treated, dosage form, administration route and schedule, but a person skilled in the art can optimize the dose level, and preferably, it can be administered at a dose of 0.001˜10 g_{PGA}/kg_{individual weight}, and more preferably, 0.01˜2 g_{PGA}/kg_{individual weight}.

If γ-PGA is added at a dose of less than 0.001 g_{PGA}/kg_{individual weight}, immune enhancement effects cannot be predicted, and if γ-PGA is added at a dose of more than 10 g_{PGA}/kg_{individual weight}, it is hard to administer the pharmaceutical composition in injectable form due to increased viscosity as well as the immune enhancement effects remains the same as dose level increased and thus it is not economical.

The inventive composition can be administered to mammals including rats, mice, domestic animals, humans and the like via various routes. The mode of administration may include, for example, oral and rectal administration, or venous, muscular, subcutaneous, endometrium or intracerebroventricular injections.

The functional food and feedstuff additive, which comprises the inventive composition for enhancing immunity containing poly-gamma-glutamic acid as an effective ingredient, can be formulated into powder, granule, tablet, capsule, liquid suspension, etc. according to the conventional methods, and a person skilled in the art can determine a preferable dose.

In another aspect, the present invention relates to a vaccine composition for preventing and treating diseases by enhancing cellular immunity, which comprises an effective dose of poly-gamma-glutamic acid.

Poly-gamma-glutamic acid according to the present invention induces enhancement of TLR mediated Th1 cellular immunity, and improves antigen presenting activity to maintain the improved activity, and thus it can be expected that it plays a role to improve effects of preventing and treating intracellular viral infection, bacterial diseases, and cancer caused by cellular mutation.

While HPV/AS04 vaccine reported to have the ability to enhance cellular immunity (Giannini S. L. et al, Vaccine. 14:5937, 2006) is available only in an injectable form, the vaccine composition of the present invention has an advantage in that it can be administered via various routes such as oral administration, nasal spray administration, etc. as well as an injectable form.

The present inventors field a patent application disclosing an adjuvant composition containing poly-gamma-glutamic acid (Korea Patent Registration No. 10-0517114), which relates to a composition which can induce high antibody titers even when antigens with poor immunogenicity are used with the composition by increasing immune response against antigen through induction of humoral immune response by the disclosed adjuvant composition.

The present invention provides a composition for enhancing cellular immunity, which comprises an effective dose of poly-gamma-glutamic acid with high molecular weight and can induce more effective cellular immune responses against antigens, which are associated with host cells or present inside the cells, and against which antibody-induced humoral immune responses are not effective. While antibodies inducing humoral immunity cannot directly penetrate into cells and thus it is hard to react with antigens inside the cells, the composition for enhancing cellular immunity according to the present invention can achieve the enhancement of cellular immunity by which antigens associated with host cells or present inside the cells, for example, antigens such as viruses, a representative example of proliferating in host cells and cancer cells generated by transformation of cells themselves can be effectively eliminated. Due to these properties, the composition is added to a vaccine composition using antigens with poor cellular immunogenicity such as killed vaccines and subunit vaccines, thus enabling the vaccine composition to have enhanced immunity effect.

### EXAMPLES

Hereinafter, the present invention will be described in more detail by examples. It is to be understood, however, that these examples are for illustrative purpose only and are not construed to limit the scope of the present invention.

### Example 1

### Production of γ-PGA and Measurement of Molecular Weight Thereof

A 5 L fermenter containing a 3 L basal medium for γ-PGA production (GS medium containing 5% L-glutamic acid: 5% glucose, 1% (NH₄)₂SO₄, 0.27% KH₂PO₄, 0.42% Na₂HPO₄.12H₂O, 0.05% NaCl, 0.3% MgSO₄.7H₂O, Vitamin solution 1 ml/L, pH 6.8) was inoculated with 1% culture broth of Bacillus subtilis var chungkookjang (KCTC 0697BP) and then cultured at a stirring speed of 150 rpm, an air injection rate of 1 vvm and a temperature of 37° C. for 72 hours. The cells were removed from the culture broth after completion of the culture using a filter press, thus obtaining a γ-PGA-containing sample solution.

2N sulfuric acid solution was added to the γ-PGA-containing sample solution and left to stand at 10° C. for 12 hours to collect a PGA precipitate. The collected precipitate was washed with a sufficient amount of distilled water to obtain γ-PGA using a Nutsche filter. The obtained poly-gamma-glutamic acid was cut with a suitable method to prepare γ-PGA having uniform molecular weight for use, or PGAs were separated according to molecular weight for use, and its molecular weight was measured using GPC (gel permeation column). In the following examples, experiments were carried out using poly-gamma-glutamic acid having an average molecular weight of 10 kDa and 2,000 kDa

### Example 2

### Toxicity Test Results Upon Oral Administration of γ-PGA

In order to examine the safety upon oral administration of γ-PGA, toxicity test upon a single oral administration of poly-gamma-glutamic acid using rats was requested to perform by Biotoxtech, an institute approved by GLP (Good Laboratory Practice) in accordance with Biotoxtech Standard Operating Procedures (SOPs), Good Laboratory Practice (GLP) regulations and test guideline.

Ten, 6-week-old male rats (159.76˜177.27 g) and ten 6-week-old female rats (121.60˜138.80 g) were used, and the dose of PGA administered to individual rats was calculated on the basis of body weight measured on the day of administration after fasting. All rats were fasted for about 16 hours but had free access to drinking water before administration, and then they were subjected to forceful oral administration with a single dose of PGA by stomach tube using a disposable syringe (5 ml) having a catheter for oral administration attached thereto, followed by being fed a feedstuff 4 hours after administration.

As a preliminary experiment, 100 mg/ml of poly-gamma-glutamic acid was orally administered to 2 male rats and 2 female rats, respectively with a single dose of 20 ml/kg, and, as a result, no dead rats were observed and thus 2000 mg/20 ml/kg was used as a single dose. An expedient was administered to a control group at the same dose as that of the experimental group to which a test material is administered. The dosage to be administered was set to 20 ml/kg.

As a result, as shown in Table 1, death and general symptoms caused by oral administration of poly-gamma-glutamic acid were not observed during the observation period. During the observation period, it was seen that the body weight of male and female rats increased in the control group and the experimental group to which a test material is administered. Autopsy results did not reveal any abnormal findings visible to the naked eye in male and female rats of the control group and the experimental group to which a test material is administered. From the result of a single oral administration of poly-gamma-glutamic acid to rats, general symptoms and death caused by the test materials were not observed so that it was determined that the fatal dose of poly-gamma-glutamic acid was more than 2000 mg/kg in female and male rats.

TABLE 1

| | Group/ | | | | | | | | | | | | | | | | | | Approximate |
|---|---|---|---|---|---|---|---|---|---|---|---|---|---|---|---|---|---|---|---|
| | Dose | No. of | Day after treatment | | | | | | | | | | | | | | | Mortality | lethal |
| Sex | (mg/kg) | animals | 1 | 2 | 3 | 4 | 5 | 6 | 7 | 8 | 9 | 10 | 11 | 12 | 13 | 14 | 15 | (dead/total) | dose (mg/kg) |
| Male | G1 | 5 | 0 | 0 | 0 | 0 | 0 | 0 | 0 | 0 | 0 | 0 | 0 | 0 | 0 | 0 | 0 | 0% | >2000 |
| | 0 | | | | | | | | | | | | | | | | | (0/5) | |
| | G2 | 5 | 0 | 0 | 0 | 0 | 0 | 0 | 0 | 0 | 0 | 0 | 0 | 0 | 0 | 0 | 0 | 0% | |
| | 2000 | | | | | | | | | | | | | | | | | (0/5) | |
| Female | G1 | 5 | 0 | 0 | 0 | 0 | 0 | 0 | 0 | 0 | 0 | 0 | 0 | 0 | 0 | 0 | 0 | 0% | >2000 |
| | 0 | | | | | | | | | | | | | | | | | (0/5) | |
| | G2 | 5 | 0 | 0 | 0 | 0 | 0 | 0 | 0 | 0 | 0 | 0 | 0 | 0 | 0 | 0 | 0 | 0% | |
| | 2000 | | | | | | | | | | | | | | | | | (0/5) | |

### Example 3

### Effect of γ-PGA on Induction of Cytokines TNF-α and IP-10 in Bone Marrow Derived Macrophages Mediated by TLR4 Signaling

In order to examine the effect of poly-gamma-glutamic acid on activation of bone-marrow derived macrophages (BMDMs) by TLR4 (Toll Like Receptor 4) signaling, the ability of γ-PGA to induce secretion of TNF-α and IP-10 (INF-γ-inducible protein) which are cytokines secreted by activated macrophages, was measured with an ELISA kit using macrophages isolated from a normal wild-type mouse (C3H/HeN, Japan SLC, Inc.) and a TLR4-mutated mouse (C3H/HeJ, Japan SLC, Inc.).

First, in order to culture bone marrow derived macrophages, long bones and short bones of 6-8 week-old female C3H/HeN and C3H/HeJ mice were taken out to cut both sides of the joints of bones, and then the bone marrow was extracted therefrom with RPMI 1640 (Gibco, USA) medium using a 5 ml syringe with a 27 G needle. Then, erythrocytes were removed using a TAC buffer (8.3 g/L ammonium chloride, 10 mM Tris-HCl, pH 7.4), and 500 μl of cell suspension was dispensed into a 24 well plate at a density of 5×10⁵ cells/well, followed by adding RPMI 1640 (added with 100 U/ml penicillin-streptomycin, 10% FBS) and M-CSF (20 ng/ml, peprotech, USA) thereto to incubate the cells at 37° C. in a CO₂ incubator for 6 days for differentiation into macrophages. At the 3rd day of culture, the same amount of M-CSF was further added and cultured.

6 days after incubation, 1 mg/ml of poly-gamma-glutamic acid (10 kDa and 2000 kDa) was diluted with RPMI 1640 at various concentrations and added to the differentiated macrophages to culture for 24 hours. At this time, a positive control group was treated with 100 ng/ml of LPS (lipopolysaccharide) which is known to promote induction of TNF-α and IP-10.

After completion of the culture, the supernatant of each well was collected, and the concentration of TNF-α present in the culture broth was measured with Mouse TNF-α ELISA kit (BD Bioscience, USA) and the concentration of IP-10 was measured with Mouse IP-10 ELISA kit (R&D, USA).

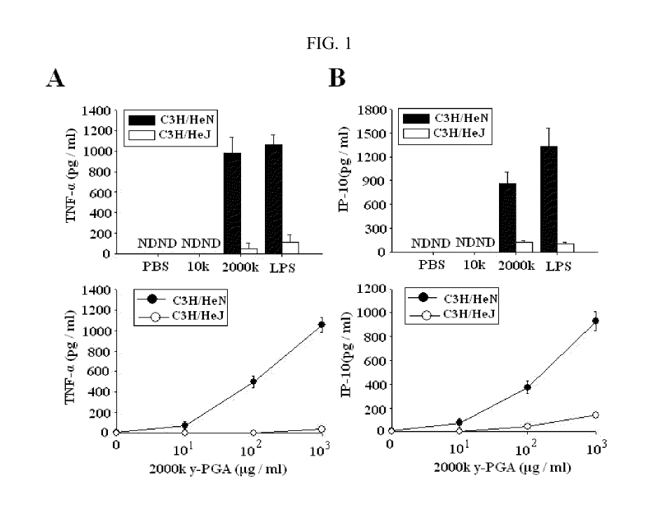

Specifically, the measurement was performed as follows, 100 μl of TNF-α or IP-10 standard solution and 100 μl of the supernatant were added into a 96 well plate coated with anti-mouse TNF-α or IP-10 monoclonal antibody and allowed to react at room temperature for 1 hour, then washed 5 times with a washing buffer (300 μl/well), followed by adding 100 μl of primary antibody, biotinylated anti-mouse TNF-α or IP-10 antibody, after which, the resulting cell suspension was allowed to react at room temperature for 1 hour and washed with a washing buffer 5 times. Then, 100 μl of secondary antibody, avidin-horseradish peroxidase conjugate was added thereto and allowed to react at room temperature for 30 minutes to wash 5 times, and then allowed to react with a color development reagent, TMB solution for 20 minutes, followed by stopping the color development with 50 μl of stop solution to measure the absorbance at 450 nm with an ELISA reader, thus analyzing the concentration of TNF-α or IP-10 by a standard curve plotted using the results obtained by the use of standard solution. A in FIG. 1 is the results obtained by measuring the amount of TNF-α secreted, and B in FIG. 1 is the results obtained by measuring the amount of IP-10 secreted.

As shown in FIG. 1, when bone marrow derived macrophages from the wild-type mouse (C3H/HeN) were treated with poly-gamma-glutamic acid having a low molecular weight of 10 kDa, cytokines were not secreted, but when the bone marrow derived macrophages were treated with poly-gamma-glutamic acid having a molecular weight of 2000 kDa and LPS, an increase in the secretion of TNF-α and IP-10 was observed, and the secreted amount of TNF-α and IP-10 was dependent on the concentration of the poly-gamma-glutamic acid having a molecular weight of 2000 kDa. The increase in TNF-a secretion indicates that enhancement of innate inflammatory immunity is induced by TLR4 mediation.

However, when bone marrow derived macrophages from TLR4 (Toll-like receptor 4) mutant C3H/HeJ mouse were treated with poly-gamma-glutamic acid (2000 kDa), it was observed that the amount of secreted cytokine decreased compared with the wild-type mouse (C3H/HeN). This result suggests that the poly-gamma-glutamic acid activates bone marrow derived macrophages through TLR4 signaling.

### Example 4

### Effect of γ-PGA on Activation of Bone Marrow-Derived Dendritic Cells by TLR4

Since dendritic cells play an important role in primary immune response as well as differentiation and activation of T-lymph, together with macrophages, the effect of poly-gamma-glutamic acid on activation of dendritic cells was examined. In addition, in order to examine the effect of TLR4 on induction of activation of bone marrow-derived dendritic cells (BMDCs) by poly-gamma-glutamic acid, bone marrow cells isolated from TLR4 wild-type C3H/HeN mouse and TLR4 mutant C3H/HeJ mouse were differentiated into dendritic cells, then used.

For differentiation into dendritic cells, bone marrow cells were dispensed into a 12 well plate at a density of 1×10⁶ cells/well to culture in a RPMI 1640 (added with 100 U/ml penicillin-streptomycin and 10% FBS) medium containing GM-CSF (Granulocyte macrophage colony-stimulating factor, 20 ng/ml) and Interleukin (IL)-4 (10 ng/ml) for 7 days, and then used.

The cultured bone marrow-derived dendritic cell suspension was diluted with poly-gamma-glutamic acid (poly-gamma-glutamic acid: 10 kDa, 2000 kDa) to a concentration of 0.1%, respectively to culture for 12 hours. At this time, a positive control group was added with 1 μg/ml of LPS (lipopolysaccharide). After completion of the culture, dendritic cells in each well were collected to examine expression of maturation surface markers on the cell surface.

In order to examine the expression of maturation surface markers, expressions of CD40, CD80 and CD86 were examined using FACSCalibur™ Flow Cytometer after the cells were stained with antibodies labeled with the respective fluorescent dyes (anti-CD40-FITC, anti-CD80-PE and anti-CD86-PE) at 4° C. for 1 hour.

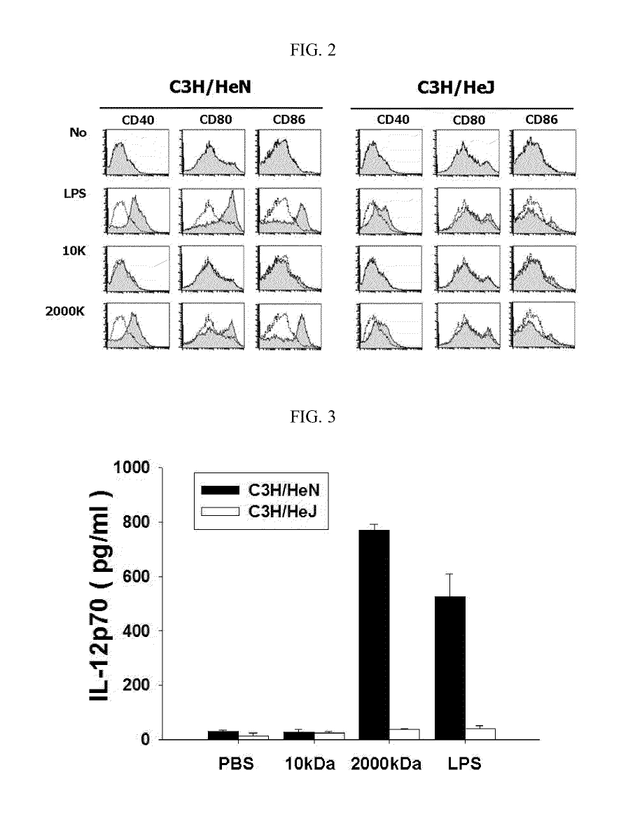

As a result, as shown in FIG. 2, from the result of the experiment using bone marrow-derived dendritic cells from TLR4 wild-type C3H/HeN mouse, it was observed that the maturation surface markers were highly expressed in the positive control group treated with LPS and the group treated with γ-PGA having a high molecular weight of 2000 kDa. However, it could be confirmed that the expression level of the maturation surface markers significantly decreased in the experiment using bone marrow-derived dendritic cells of TLR4 mutant C3H/HeJ mouse.

### Example 5

### Effect of γ-PGA on Induction of IL-12 Secretion in Bone Marrow-Derived Dendritic Cells by TLR4

As described in Example 3, bone marrow cells isolated from TLR4 wild-type C3H/HeN mouse and TLR4 mutant C3H/HeJ mouse were differentiated into dendritic cells, and the cell suspension was diluted with poly-gamma-glutamic acid (poly-gamma-glutamic acid: 10 kDa, 2000 kDa) to a concentration of 0.1%, respectively to culture for 24 hours, and then, each culture supernatant was collected to measure IL-12p70 concentration in the culture supernatant using Mouse IL-12p70 ELISA kit (BD biosciences, USA).

100 μl of IL-12p70 standard solution and 100 μl of the culture supernatant were added into a 96 well plate coated with anti-mouse IL-12p70 monoclonal antibody and allowed to react at room temperature for 2 hours to wash 3 times with a washing buffer (300 μl/well). Then, the resulting cell suspension was added with 100 μl of primary antibody, biotinylated anti-mouse IL-12p70 polyclonal antibody and allowed to react at room temperature for 2 hours to wash 3 times with a washing buffer (300 μl/well). After that, 100 μl of secondary antibody, streptavidin-horseradish peroxidase conjugate was added thereto and allowed to react at room temperature for 1 hour to wash 5 times, and then allowed to react with a color development reagent, TMB solution for 30 minutes, followed by stopping the color development with 50 μl of stop solution to measure the absorbance at 450 nm with an ELISA reader, thus analyzing the amount of IL-12p70.

As a result, as shown in FIG. 3, poly-gamma-glutamic acid (2000 kDa) induced high level of IL-12p70 secretion in bone marrow-derived dendritic cells from TLR4 wild-type C3H/HeN mouse, like the positive control group treated with LPS. However, it was found that the level of IL-12p70 secretion decreased significantly in bone marrow-derived dendritic cells from TLR4 mutant C3H/HeJ mouse.

As shown in FIG. 2 and FIG. 3, it could be confirmed that poly-gamma-glutamic acid could activate dendritic cells and induce an IL-12 cytokine involved in enhancement of cellular immunity, and as can be seen in macrophages, dendritic cell activation was molecular weight dependent. Moreover, from the fact that dendritic cell-activating factors significantly increased in wild-type mouse, were significantly decreased in TLR4-mutated mouse, it could be seen that poly-gamma-glutamic acid activates dendritic cells by TLR4 signaling in the same manner as LPS used in the positive control.

### Example 6

### Effect of γ-PGA on the Improvement of Antigen Presenting Activity in Dendritic Cells

Dendritic cells, which are specialized antigen-presenting cells, activate T cells by presenting antigens to T cells by cell-surface MHC molecules. In order to examine whether poly-gamma-glutamic acid increases antigen presenting activity in dendritic cells, mixed lymphocyte reaction was performed.

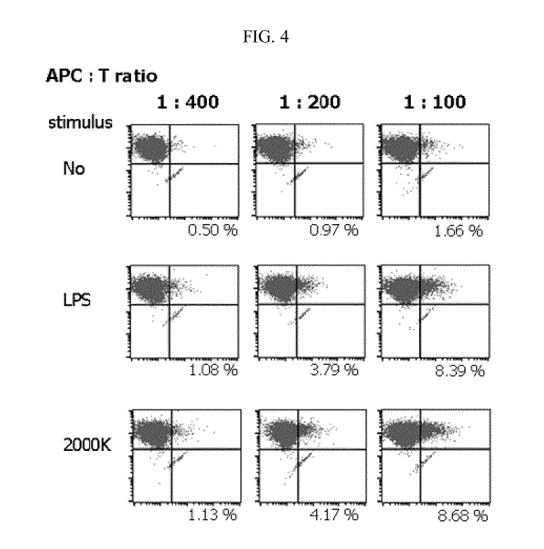

Dendritic cells, differentiated from mouse bone marrow cells, were inoculated to a density of 1×10⁶ cells/ml, and stimulated with LPS (1 μg/ml) or poly-gamma-glutamic acid of 2000 kDa (1 mg/ml) for 12 hours, then treated with 1 μg/ml of MHC class I-restricted E7 peptide (aa 49-57, AniGen, Korea) for 1 hour to wash 2 times with a RPMI-1640 medium. The thus obtained dendritic cells were mix-cultured with HPV16 E7 aa 49-57 peptide-specific T cells at the ratio indicated in FIG. 4 in a medium containing 1 μg/ml of GolgiPlug at 37° C. overnight.

In order to examine activated T cells, distribution of CD8+ T cells secreting IFN-γ was analyzed by FACS. Specifically, after the cells were washed, and then stained with an antibody specific for T-cell surface molecule CD8 (PE-conjugated anti-mouse CD8) at 4° C. for 1 hour, intracellular IFN-γ was stained with an antibody specific for IFN-γ (FITC conjugated anti-mouse IFN-γ) using Cytofix/Cytoperm kit (BD Bioscience, USA).

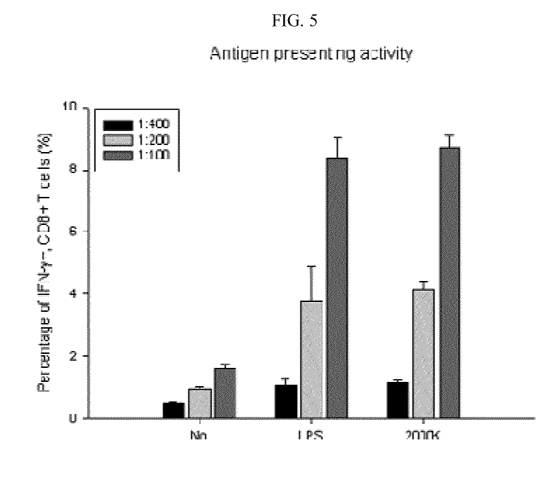

As shown in FIG. 4, it could be confirmed that cells without stimulation could not increase T cell activity when the ratio between dendritic cells to T cells was increased, while dendritic cells treated with poly-gamma-glutamic acid (2000 kDa) significantly increased T cell activity as the ratio between dendritic cells to T cells was increased. Particularly, when the dendritic cells and the T cells were mix-cultured at a ratio of 1:100, the result showed that activated T cells were 8.68±0.4%, which is high value and similar to 8.39±0.6% resulted from the positive control group treated with LPS. Thus, it was confirmed that poly-gamma-glutamic acid increases antigen presenting activity in dendritic cells to induce T cell activity (FIG. 5).

### Example 7

### Examination on a Decrease in TLR4-Dependent Signaling by γ-PGA Through Inhibition of TLR4 Gene Expression Using siRNA

Control siRNA (catalog no. D-001210-0, Dharmacon, USA) and siTLR4 (catalog no. M-047487-00, Dharmacon, USA) were diluted in 500 μA of 5× siRNA buffer, respectively, and each 50 μl of the diluent was dispensed and stored at −20° C. For transient transfection, RAW 264.7 cells (KCLB 40071) were cultured in a 100φ culture dish to a density of 2×10⁵ cells/ml. The cultured cells were collected using 0.5% Trypsin-EDTA and washed 3 times with PBS. The obtained cells were subjected to transient transfection with siTLR4 or control siRNA by electroporation. The cells were suspended in 20 μl of electroporation buffer, and each 10 μl of suspension was dispensed into two tubes, then added with each 10 μl of control siRNA and siTLR4 and placed on ice for 5 minutes. After being placed on ice for 5 minutes, electroporation (2 pulses, 1230 voltage) was performed. The transient transfected cells were suspended in DMEM culture broth containing 10% FBS.

First, inhibition of TLR4 mRNA expression by siTLR4 was examined using RT-PCR. RAW264.7 cells were subjected to transient transfection with control siRNA and siTLR4 to culture for 48 hours, and cells were collected. The collected cells were washed 2 times with PBS, and total RNA was obtained using RNeasy Mini kit (Qiagen). Using 2 μg of the obtained total RNA, cDNA was synthesized by M-MLV reverse transcriptase (Promega, USA). The synthesized cDNA was amplified using the following primers: sense; 5′-GCA TGG CTT ACA CCA CCT CT-3′ (SEQ ID NO: 1), antisense; 5′-GTG CTG AAA ATC CAG GTG CT-3′ (SEQ ID NO: 2)) which specifically recognize TLR4 and the PCR amplication conditions consisted of 30 cycles of 95° C. for 60 sec, 65° C. for 60 sec and 72° C. for 80 sec. As a positive control, the following primers: sense; 5′-GCT ACA GCT TCA CCA CCA CA-3′ (SEQ ID NO: 3), antisense; 5′-CTA CGT ACT CCT GCT TGC TG-3′ (SEQ ID NO: 4) which recognize β-actin were used to amplify cDNA under the PCR conditions consisting of 30 cycles of 94° C. for 45 sec, 56° C. for 45 sec and 72° C. for 45 sec. The amplified PCR products were electrophoresed in a 1.2% agarose gel to examine the expression of TLR4 (950 bp) and β-actin (450 bp).

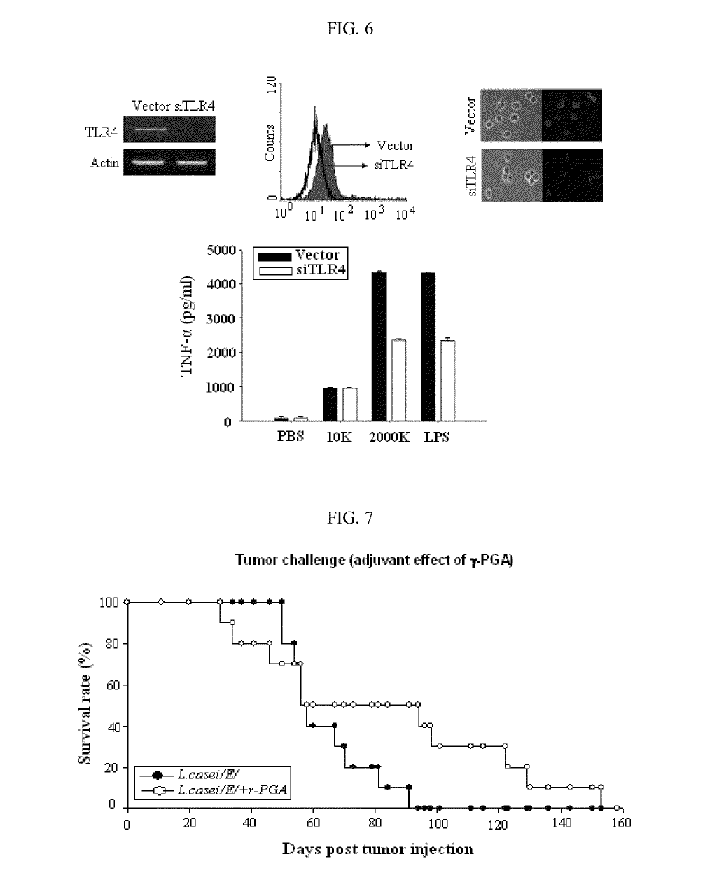

As a result, it could be seen that the expression of TLR4 mRNA significantly decreased in siTRL4-transfected cells, while TLR4 mRNA was normally expressed in cells transfected with control siRNA (FIG. 6).

In order to examine inhibition of protein expression, immunofluorescence assay and flow cytometric analysis using FACSCaliber were performed. For the immunofluorescence assay, RAW 264.7 cells were subjected to transient transfection with control siRNA and siTLR4 to culture for 48 hours, and cells were collected. The collected cells were attached to a cover glass to a density of 2×10⁴ cells/ml. The cells attached to the cover glass were washed 2 times with PBS, and added with 2% BSA dilution dissolved in TBS-T (a buffer solution, TBS containing 0.05% Tween-20), then left to stand at room temperature for 1 hour. Anti-TLR4 antibody (Santa cruz, USA) was diluted 1:100 in a 2% BSA dilution and allowed to react at room temperature for 2 hours to wash 3 times with TBS-T buffer solution. After washing, FITC (fluorescein isothiocyanate)-conjugated IgG antibody (Sigma, USA) was diluted 1:200 in a 2% BSA dilution and left to stand at room temperature for 30 minutes to wash 3 times with TBS-T buffer solution, then fluorescence of cells was observed using Axioskop inverse fluorescence microscope (Carl Zeiss, German). As a result of immunofluorescence assay, it could be seen that fluorescence was significantly decreased in siTRL4-transfected cells while fluorescence was detected on the surface of all control siRNA-transfected cells observed under the microscope. Therefore, it could be confirmed that siTLR4 inhibited TLR4 protein expression (FIG. 6).

For FACS analysis, RAW264.7 cells were subjected to transient transfection with control siRNA and siTLR4 to culture for 48 hours to collect cells. The collected cells were washed 2 times with PBS, and added with 2% BSA dilution dissolved in TBS-T (a buffer solution, TBS containing 0.05% Tween-20), then left to stand at room temperature for 1 hour. Anti-TLR4 antibody was diluted 1:100 in a 2% BSA dilution and allowed to react at room temperature at 4° C. for 1 hour to wash 3 times with TBS-T buffer solution. After washing, FITC-conjugated IgG antibody was diluted 1:200 in a 2% BSA dilution and left to stand at 4° C. for 30 minutes to wash with TBS-T buffer solution 3 times, then analyzed for fluorescence intensity by FACS Caliber (BD Bioscience, USA). As a result, it could be confirmed that the expression of TLR4 receptor was inhibited by siTLR4, like fluorescence microscopy analysis (FIG. 6).

In order to examine whether poly-gamma-glutamic acid induces intracellular TLR4 signaling, TLR4 gene expression was inhibited using siRNA to measure intracellular secretion of TLR4-dependent cytokine TNF-α.

RAW264.7 cells were subjected to transient transfection with control siRNA and siTLR4 to culture for 48 hours, and stimulated with LPS (100 ng/ml), poly-gamma-glutamic acid of 10 kDa (1 mg/ml), and poly-gamma-glutamic acid of 2000 kDa (1 mg/ml) to culture for 24 hours. After stimulating for 24 hours, culture broth was collected to measure secreted TNF-a with ELISA kit (BD Bioscience, USA).

First, TNF-α capture antibody was diluted 1:250 in a 96-well plate and left to stand at 4° C. for 16 hours. The plate coated with TNF-a capture antibody was added with assay diluent and left to stand at room temperature for 1 hour to inhibit nonspecific reactions. After inhibition of the nonspecific reactions, the antibody coated plate was washed 3 times with a washing solution, and each 100 μl of culture broth was added to each well, then left to stand at 37° C. for 2 hours. After the addition of the cell culture broth, 100 μl of mixture solution of TNF-a detection antibody and avidin HRP (Horseradish peroxidase-conjugated) antibody (1/250 dilution), was added to each well and left to stand at 37° C. for 2 hours to wash 3 times. After washing, 100 μl of TMB substrate (color development reagent) was added to each well and left to stand at room temperature for 30 minutes to develop color, thus measuring the absorbance at 450 nm.

As a result, as shown in FIG. 6, it could be confirmed that TNF-a concentration was significantly increased in the cell culture broth treated with LPS and poly-gamma-glutamic acid of 2000 kDa after control siRNA transfection, compared with a control group (cells treated with PBS), while TNF-a concentration was significantly decreased in the cell culture broth treated with LPS and poly-gamma-glutamic acid of 2000 kDa after transfected with siTLR4, compared with the cell culture broth transfected with control siRNA. However, TNF-α concentration in the cell culture broth treated with poly-gamma-glutamic acid of 10 kDa after control siRNA transfection, was increased compared with the control group, but no difference in TNF-α concentration was shown in the case of siTLR4 transfection. Thus, similar results were obtained in examining the effect of LPS known as TLR4 ligand and poly-gamma-glutamic acid of 2000 kDa on the secretion of TLR4-dependent cytokine TNF-α, in cells in which TLR4 gene expression is inhibited by siTLR4, suggesting that the poly-gamma-glutamic acid of 2000 kDa induces TLR4-dependent signaling.

### Example 8

### Examination on Improvement in Cancer Treatment Effects of Lactobacillus Having HPV Antigen Protein Expressed on the Surface Thereof by Poly-Gamma-Glutamic Acid

In the case of using microorganisms expressing an antigen protein on their surface as a vaccine, whether poly-gamma-glutamic acid having a high molecular weight plays a role as an adjuvant for cell mediated immune responses (CMI-adjuvant) to improve antigen-specific anticancer effects, was examined.

Using an animal model for human papillomavirus (HPV)-induced cervical cancer, Lactobacillus having an antigen protein E7 expressed on the surface thereof was used as a main antigen component of a therapeutic vaccine, and cell-mediated immunity enhancement effects were compared using two experimental groups according to administration of Lactobacillus having E7 expressed on the surface thereof with or without poly-gamma-glutamic acid.

Using pgsA among genes (pgsA, pgsB and pgsC) of outer membrane protein derived from Bacillus sp., which is involved in synthesizing poly-gamma-glutamic acid, gram positive and gram negative microorganisms were used as a host to construct a vector pHCE2LB: pgsA-HPV-E7 capable of surface expressing HPV-specific antigen E7, thus obtaining Lactobacillus having E7 expressed on the surface thereof by transforming the constructed vector into Lactobacillus casei.

The pHCE2LB: pgsA-HPV-E7 construct surface expressing antigen protein E7 was prepared as follows. PCR reaction was performed using a DNA fragment encoding HPV-E7 among human papillomavirus genomic DNAs (GenBank accession No. K02718), as a template (SEQ ID NO: 5), with primers of SEQ ID NO: 6 (5′-cgcggatccatgcatggagatacacc-3′) and SEQ ID NO: 7 (5′-cgctctagattatggtttctgagaacag-3′) to obtain a 315 by fragment and the obtained DNA fragment was digested with BamHI and XbaI restriction enzymes to obtain insert fragments. The insert fragments were inserted into HPVL1 of pHCE2LB: pgsA-HPVL1 (KCTC 10349BP: Escherichia coli transformed with pHCE2LB: pgsA-HPVL1) digested with BamHI and XbaI restriction enzymes to construct pHCE2LB: pgsA-HPV-E7.

In order to express E7 protein on the cell surface, the surface expression vector, pHCE2LB:pgsA-HPV-E7 was transformed into Lactobacillus casei to construct a transformant expressing E7 on the surface thereof, and the transformant was stationary cultured at 37° C. in a MRS medium (Lactobacillus MRS, Becton Dickinson and Company Sparks, USA) to induce surface expression.

Using C57BL/6 mice (Dae Han Biolink Co., Ltd., Korea) injected with cancer cells expressing E7, TC-1 (ATCC CRL-2785), survival rate of the cancer cell-injected mice orally administered the constructed Lactobacillus expressing E7 antigen and poly-gamma-glutei acid, and the cancer cell-injected mice orally administered only with Lactobacillus expressing E7 antigen, was examined.

First, ten 6-8-week-old female C57BL/6 mice were prepared for each group, and 2×10⁴ TC-1 cancer cells were subcutaneously injected (s.c.) into the inner side of the left thigh to induce cancer. Starting from 3 days after cancer cell injection, Lactobacillus having E7 antigens expressed on the surface thereof (2×10⁹) and a mixture of Lactobacillus and 400 μg of poly-gamma-glutamic acid of 2000 kDa were orally administered 5 times per week for 2 weeks, and the first boost was given after a rest period of one week and the second boost was given after another rest period of one week.

As a result, as shown in FIG. 7, it was confirmed that all mice died within 90 days after cancer cell injection in the test group orally administered only Lactobacillus having E7 antigens expressed on the surface thereof, while all mice died within 150 days after cancer cell injection in the test group orally administered the mixture of Lactobacillus and y-PGA suggesting that y-PGA increased survival rate. Thus, it could be confirmed that poly-gamma-glutamic acid plays a role as an adjuvant for cell-mediated immune responses (CMI-adjuvant) of oral vaccines to improve anticancer effects against a specific antigen.

### Example 9

### Cancer Treatment Effects of a Composition Containing Poly-Gamma-Glutamic Acid and Lactobacillus Having Carcinoembryonic Antigen CEA Expressed on the Surface Thereof

Lactobacillus expressing CEA (carcinoembryonic antigen) on the surface thereof used as a main antigen component of a vaccine was added with poly-gamma-glutamic acid having a high molecular weight as an adjuvant for cell mediated immune responses (CMI-adjuvant) to examine anticancer effects.

To obtain a construct capable of expressing CEA on the surface thereof, PCR reaction was performed using 1683 by DNA (SEQ ID NO: 8) containing human CEA gene, as a template, with primers of SEQ ID NO: 9 (5′-cgcagatctccggagctgcccaagccc-3′) and SEQ ID NO: 10 (5′-cgctctagatcatatcagagcaaccccaacc-3′), to obtain a 1704 by DNA fragment, and the obtained fragment was digested with BglII and XbaI restriction enzymes, to obtain insert fragments. The insert fragments were inserted into HPVL1 of pHCE2LB: pgsA-HPVL1 (KCTC 10349BP: Escherichia coli transformed with pHCE2LB: pgsA-HPVL1) digested with BamHI and XbaI restriction enzymes to construct pHCE2LB: pgsA-hCEA as described in Example 8.

In order to express CEA protein on the cell surface, the surface expression vector, pHCE2LB:pgsA-hCEA was transformed into Lactobacillus casei to construct a transformant expressing hCEA on the surface thereof, and the transformant was stationary cultured at 37° C. in a MRS medium (Lactobacillus MRS, Becton Dickinson and Company Sparks, USA) to induce surface expression.

### 1) Humoral Immune Response

Lactobacillus casei having CEA expressed on the surface thereof and poly-glutamic acid were orally and nasally administered to mice for a given period, and then a humoral immune response against CEA in mouse blood was examined before subcutaneous injection of cells overexpressing CEA.

2 groups, each group consisting of fifteen female 5-week-old C57BL/6 mice (Dae Han Biolink Co., Ltd., Korea) were prepared as an experimental group and a control group, and a mixture of 1×10¹¹ Lactobacillus casei having CEA expressed on the surface thereof and 100 μl of 1% poly-gamma-glutamic acid (a molecular weight of 7000 kDa) was orally administered as a single daily dose to the mice of the experimental group for 5 days in the 1st week and 5 days in the 2nd week (10 days total), and booster was given for 5 days in the 4th week and 6th week, respectively. In the case of nasal administration, a mixture of 1×10¹⁰ Lactobacillus casei having CEA expressed on the surface thereof and 10 μl of 1% poly-gamma-glutamic acid (a molecular weight of 7000 kDa) was administered as a single daily dose to the test animals at a 2-day interval in the 1st week and the 2nd week (4 days total), and booster was given for 2 days in the 4th week and 6th week, respectively.

Preimmune serum was taken from each mouse before administration of Lactobacillus casei having CEA expressed on the surface thereof, and serum was taken from each mouse on the third week (primary serum), on the fifth week (secondary serum) and on the seventh week (tertiary serum) to analyze humoral immunity effect. Mice in the control group were administered Lactobacillus casei, on the surface of which CEA is not expressed, with a dose of 1×10¹¹/day for the same period as that of the experimental group.

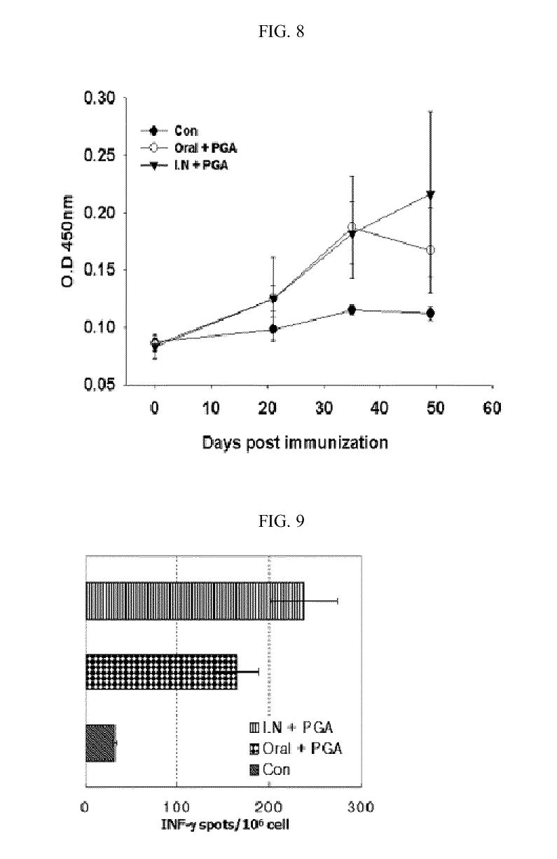

Whether humoral immunity against CEA is induced in mice of the experimental group and in mice of the control group, was examined with an ELISA method. First, a 96-well Nunc-immune plate was coated with CEA antigen (Fitzgerald, USA). The antigen diluted in a 0.1M bicarbonate coating buffer (pH 9.6) to a concentration of 100 ng/100 μl was added into each well and left to stand at 4° C. overnight, thus coating each well with CEA. The CEA-coated plate was washed 3 times with PBST, blocked with 10% skim milk dissolved in PBS (a blocking solution) for 2 hours, allowed to react with a primary antibody (mouse serum) at 37° C. for 2 hours, and allowed to react with a secondary antibody conjugated to peroxidase for 1 hour. In each step, the plate was washed 5 times using PBS-T. Then, TMB substrate solution (BD Biosciences, USA) was added into each well and allowed to react at room temperature for 20 minutes to stop the reaction with a 0.2N H₂SO₄ stop solution, then the absorbance was measured at 450 nm with ELISA reader to measure the IgG antibody titer to AFP in mouse serum (FIG. 8).

As a result, as shown in FIG. 8, it could be confirmed that the IgG antibody titer to CEA in serum of the control group had no significant change during administration period, while the IgG antibody titer to CEA in the experimental group increased during the administration period. From the above results, it was confirmed that humoral immune response against human CEA was induced in mice by the composition containing Lactobacillus having human CEA expressed on the surface thereof and poly-gamma-glutamic acid.

### 2) Cell-Mediated Immune Response

Lactobacillus casei having CEA expressed on the surface thereof and poly-glutamic acid were orally and nasally administered to mice for a given period, and then mouse splenic T-cell immune response to CEA was examined.

In order to examine a cellular immune response to CEA expressed on the surface of Lactobacillus casei, first, 5 mice were selected among 15 mice of the group in the above 1), and spleens were taken from each mouse and placed into tubes containing 15 ml of RPMI medium (Gibco BRL, USA). The spleen was transferred into a sterile petri dish, and spleen and thymus cell suspensions were made by squeezing the organs between 2 sterile glass slides. The cell suspension in the petri dish was transferred to a 50 ml tube containing RPMI medium, and the tube containing the sell suspension was placed on ice for 15-20 minutes, then 40-45 ml of supernatant was transferred to a new 50 ml tube. Each tube was centrifuged at 1600-2000 rpm for 10 minutes, and pellets, from which the supernatant was removed, were added with 10 ml of preheated TAC buffer [10 mM NH₄Cl, Tris-Cl (pH7.4)] to suspend cells, then left to stand in a water bath maintained at 37° C. for 10 minutes to hemolyze erythrocytes. The hemolyzed erythrocytes were removed by stirring the cells with a glass pipette to have them attached to the pipette and the tube was filled with RPMI medium. The cells in the tube were washed 2 times with RPMI medium and suspended in RPMI 1640 medium to isolate splenocytes.

A cell-mediated immune response to CEA was examined by the following two methods, using splenocytes, isolated from the mice immunized with Lactobacillus casei having CEA expressed on the surface thereof and γ-PGA, and non-treated mice.

\textcircled{1} ELISPOT Assay

ELISPOT assay was first performed among measurement methods of cellular immune response. ELISPOT plate (BD ELISPOT Mouse IFN-γ Set, USA) was coated with IFN-γ specific antibody diluted 1:200 in PBS (pH 7.2), and left to stand overnight. 100 μl of the isolated splenocytes and thymic cells were dispensed into each well at a density of 2×10⁶ cells/ml and allowed to react for 2 hours, and then 7 μl/ml of CEA Peptide [EAQNTTYL] (AnyGen, Korea) was added into each well and allowed to react at 37° C. under conditions of 5% CO₂ for 48 hours. After the reaction, each well was washed 5 times with PBS-T, and 100 μl of biotinylated secondary antibody (BD Biosciences, USA) diluted 1:250, was dispensed into each well, and then allowed to react at room temperature for 2 hours. Then, each well was washed 5 times with PBS-T, and 100 μl of enzyme conjugate (streptavidin-HRP) reagent (BD Biosciences, USA) diluted 1:200 was added into each well, then allowed to react for 1 hour, followed by washing again 5 times with PBS-T and PBS to analyze the number of spots appeared after color development with a substrate solution (BD AEC Substrate Reagent Set, USA) using an ELISPOT plate reader (BD Biosciences, USA) (FIG. 9).

As a result, as shown in FIG. 9, it was observed that the number of spots in the experimental group was significantly increased compared with the control group. From the results, it could be confirmed that splenic T-cell immunity to CEA was activated in mice administered Lactobacillus casei having CEA expressed on the surface thereof and PGA.

\textcircled{2} Intracellular Cytokine Staining (IFN-γ Staining)

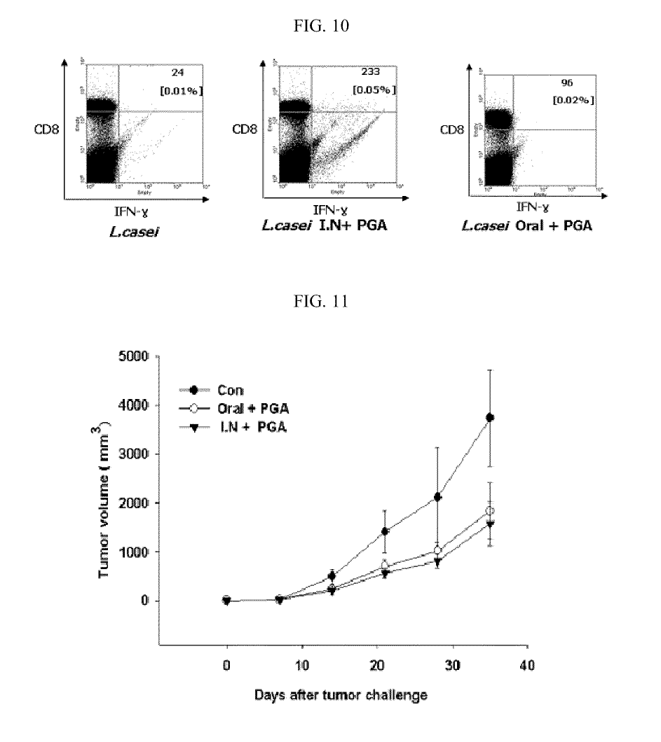

The isolated splenocytes were dispensed into a 24 well plate to a density of 3×10⁶ cells/well and allowed to react at 37° C. under conditions of 5% CO₂ for 2 hours, then 7 μg/ml of CEA peptides [EAQNTTYL] (AnyGen, Korea) was dispensed into each well and allowed to react for 2 hours. After the reaction, 4 μl of Golgi stop solution (BD Golgi STOP, USA) was added into each well, and allowed to react at 37° C. under conditions of 5% CO₂ for 9 hours. The cells in each well, stimulated with peptides, were centrifuged at 1600 rpm for 5 minutes, and CD8-PE antibodies (Phycoerythrin; BD Biosciences, USA) were diluted 1:200 in FACS staining buffer (DPBS, 1% FBS, 0.09% sodium azide) to mix with cells, then allowed to react at 4° C. for 30 minutes. Each well was washed 2 times with FACS staining buffer and added with 200 μl of Fixation/Permeablization kit (BD Cytofix/Cytoperm, USA), then allowed to react at 4° C. for 20 minutes, after that, each well was washed 2 times with BD perm washing buffer (BD Cytofix/Cytoperm, USA), and IFN-γ-FITC antibodies (BD Biosciences, USA) were diluted 1:200 in BD perm washing buffer to mix with cells, then allowed to react at 4° C. for 30 minutes to wash 2 times, followed by suspending cells in 300 μl of FACS staining buffer to analyze using FACS Caliber (Becton Dickinson and company) (FIG. 10).

As a result, as shown in FIG. 10, it was confirmed that IFN-γ, which is a cytokine involved in anticancer activity, was expressed at high levels in mouse splenic CD8+ T cells, involved in cell-mediated immunity, of the experimental group compared to the control group.

As described above, it was confirmed through the above two methods, ELISPOT assay and intracellular cytokine staining (IFN-γ staining) that the mixture composition of Lactobacillus having CEA expressed on the surface thereof and poly-gamma-glutamic acid induced cellular immune response to CEA by stimulating mouse splenic T cells.

### 3) Protective Anti-Cancer Immunity Effects of a Mixture Composition of Microorganisms Having CEA Expressed on the Surface Thereof and Poly-Gamma-Glutamic Acid on Subcutaneous Colon Cancer Model.

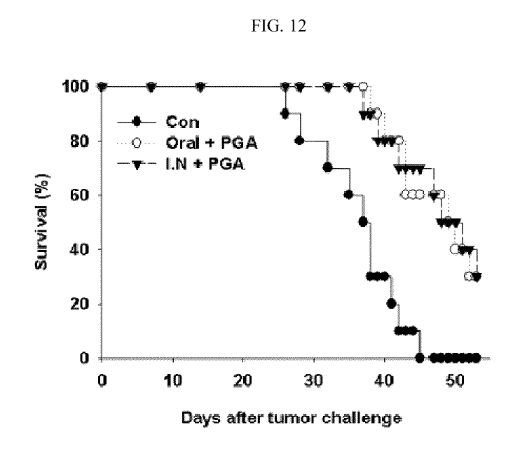

5×10⁶ of MC38 CEA2 cells (Robbins P F et al. Cancer research 51:3657, 1991), which overexpress CEA, were injected subcutaneously into the left femoral region of each 10 mice of the experimental group and the control group as described in the above 1) using a 30 gauge needle for 8 weeks after cancer cell injection, mice were examined for cancer formation and death rate (FIG. 11 and FIG. 12).

As a result, as shown in FIG. 11, it was found that, on the 5th week after cancer cell injection, mice in the control group showed cancer cell proliferation area larger than 3500 mm³, while mice in the experimental group, which was orally and nasally administered Lactobacillus having CEA express on the surface thereof and PGA, showed cancer cell proliferation area of about 1500 mm³.

In addition, as shown in FIG. 12, as a result of measuring mortality rate more than 50 days after cancer cell injection, it was found that mice in the control group began to die on the 30th day after cancer cell injection, thereafter rapid death was observed, while mice in the experimental group began to die on the 40th day, and relatively slow death was observed compared to the control group. Moreover, on the 43th day, the mice in the control group showed a mortality of 100%, while the mice in the experimental group showed a survival rate of more than 40%. The above results suggested that cancer cell proliferation in mice was inhibited by the vaccine composition comprising a mixture of Lactobacillus having CEA on the surface thereof and poly-gamma-glutamic acid

### INDUSTRIAL APPLICABILITY

As described and demonstrated above in detail, the inventive composition comprising an effective dose of poly-gamma-glutamic acid has very little toxicity and side effects, and can be added to a vaccine composition for preventing and treating animal viral or bacterial infections and cancer, or a vaccine composition for preventing and treating human viral or bacterial infections and cancer to show cellular immunity-enhancing effects.

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

1. A method for enhancing cellular immunity, which comprises a step of administering a composition comprising an effective dose of poly-gamma-glutamic acid.

2. The method according to claim 1, wherein the enhancement of cellular immunity is mediated by TLR (Toll-like receptor).

3. The method according to claim 1, wherein the average molecular weight of poly-gamma-glutamic acid is 100 kDa˜15,000 kDa.

4. The method according to claim 1, which comprises a pharmaceutically acceptable carrier.

5. A method for preventing or treating viral or bacterial infections, which comprises a step of administering a vaccine composition comprising an effective dose of poly-gamma-glutamic acid.

6. The method according to claim 5, wherein said viral or bacterial infections are human or animal infection.

7. A method for preventing or treating cancer, which comprises a step of administering a vaccine composition comprising an effective dose of poly-gamma-glutamic acid.


SEQUENCE LISTING (10 sequences)

SEQ ID NO: 1 - DNA, 20 bp, Artificial Sequence
  Synthetic Construct
gcatggcttacaccacctct

SEQ ID NO: 2 - DNA, 20 bp, Artificial Sequence
  Synthetic Construct
gtgctgaaaatccaggtgct

SEQ ID NO: 3 - DNA, 20 bp, Artificial Sequence
  Synthetic Construct
gctacagcttcaccaccaca

SEQ ID NO: 4 - DNA, 20 bp, Artificial Sequence
  Synthetic Construct
ctacgtactcctgcttgctg

SEQ ID NO: 5 - DNA, 297 bp, Human papillomavirus
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 6 - DNA, 26 bp, Artificial Sequence
  Synthetic Construct
cgcggatccatgcatggagatacacc

SEQ ID NO: 7 - DNA, 28 bp, Artificial Sequence
  Synthetic Construct
cgctctagattatggtttctgagaacag

SEQ ID NO: 8 - DNA, 1683 bp, Homo sapiens
ccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccatgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgata

SEQ ID NO: 9 - DNA, 27 bp, Artificial Sequence
  Synthetic Construct
cgcagatctccggagctgcccaagccc

SEQ ID NO: 10 - DNA, 31 bp, Artificial Sequence
  Synthetic Construct
cgctctagatcatatcagagcaaccccaacc